(19) United States
(12) Patent Application Publication
(10) Pub. No.: US 2025/0339012 A1
(43) Pub. Date: Nov. 6, 2025

(54) TELE-ENDOSCOPY

(71) Applicant: MicronVision Corp., Bellevue, WA (US)

(72) Inventor: Xiaolong OUYANG, Bellevue, WA (US)

(21) Appl. No.: 19/272,556

(22) Filed: Jul. 17, 2025

Related U.S. Application Data
(63) Continuation-in-part of application No. 19/089,980, filed on Mar. 25, 2025.
Application No. 19/089,980 is a continuation-in-part of application No. 18/628,133, filed on Apr. 5, 2024.
Application No. 18/628,133 is a continuation-in-part of application No. 18/083,209, filed on Dec. 16, 2022.
Application No. 18/083,209 is a continuation-in-part of application No. 17/941,884, filed on Sep. 9, 2022.
Application No. 19/089,980 is a continuation-in-part of application No. 18/212,621, filed on Jun. 21, 2023.
Application No. 18/212,621 is a division of application No. 17/720,143, filed on Apr. 13, 2022, now Pat. No. 11,771,304.
Application No. 17/720,143 is a continuation-in-part of application No. 17/521,397, filed on Nov. 8, 2021, now Pat. No. 11,980,342.
(60) Provisional application No. 63/112,739, filed on Nov. 12, 2020.
(60) Provisional application No. 63/113,960, filed on Nov. 15, 2020.
(60) Provisional application No. 63/118,617, filed on Nov. 25, 2020.
(60) Provisional application No. 63/128,105, filed on Dec. 20, 2020.
(60) Provisional application No. 63/138,528, filed on Jan. 18, 2021.
(60) Provisional application No. 63/176,307, filed on Apr. 18, 2021.
(60) Provisional application No. 63/256,634, filed on Oct. 18, 2021.
(60) Provisional application No. 63/282,108, filed on Nov. 22, 2021.
(60) Provisional application No. 63/283,367, filed on Nov. 26, 2021.
(60) Provisional application No. 63/295,913, filed on Jan. 2, 2022.
(60) Provisional application No. 63/299,829, filed on Jan. 14, 2022.
(60) Provisional application No. 63/299,960, filed on Jan. 15, 2022.
(60) Provisional application No. 63/302,563, filed on Jan. 25, 2022.
(60) Provisional application No. 63/303,690, filed on Jan. 27, 2022.
(60) Provisional application No. 63/310,336, filed on Feb. 15, 2022.
(60) Provisional application No. 63/332,233, filed on Apr. 18, 2022.
(60) Provisional application No. 63/417,340, filed on Oct. 19, 2022.
(60) Provisional application No. 63/672,949, filed on Jul. 18, 2024.
(60) Provisional application No. 63/683,198, filed on Aug. 14, 2024.
(60) Provisional application No. 63/685,682, filed on Aug. 21, 2024.
(60) Provisional application No. 63/693,589, filed on Sep. 11, 2024.
(60) Provisional application No. 63/713,926, filed on Oct. 30, 2024.
(60) Provisional application No. 63/718,614, filed on Nov. 9, 2024.
(60) Provisional application No. 63/734,073, filed on Dec. 14, 2024.
(60) Provisional application No. 63/743,649, filed on Jan. 10, 2025.
(60) Provisional application No. 63/756,997, filed on Feb. 11, 2025.
(60) Provisional application No. 63/766,315, filed on Mar. 3, 2025.

(51) Int. Cl. A61B 1/00; A61B 1/015; A61B 1/018; A61B 1/05; A61B 17/02; A61B 17/34; G16H 30/40

(52) U.S. Cl. CPC A61B 1/00032; A61B 1/00131; A61B 1/015; A61B 1/018; A61B 1/05; A61B 17/0218; A61B 17/3421; G16H 30/40

(57) ABSTRACT

A tele-endoscopy system and method enable low-cost hand-held endoscopes to be used both (i) when linked so that a local endoscope controls a remote endoscope and (ii) individually so that a remote endoscope can carry out or continue a procedure even if the link to the remote endoscope degrades or there is no link. The system fills a gap between expensive telesurgery that critically depend on a high quality communication link and individually used endoscopes and is particularly intuitive and user-friendly.

### REFERENCE TO RELATED APPLICATIONS

This application is a continuation-in-part of U.S. patent application Ser. No. 19/089,980 filed Mar. 25, 2025, which is a continuation in-part of U.S. patent application Ser. No. 18/628,133 filed Apr. 5, 2024, which is a continuation-in-part of U.S. patent application Ser. No. 18/083, 209 filed Dec. 16, 2022, which is a continuation-in-part of U.S. patent application Ser. No. 17/941,884 filed Sep. 9, 2022, which claim priority to U.S. Provisional Appln. Ser. Nos. 63/417,340 filed Oct. 19, 2022; 63/256,634 filed Oct. 18, 2021; 63/282,108 filed Nov. 22, 2021; 63/283,367 filed Nov. 26, 2021; and 63/332,233 filed Apr. 18, 2022.

This application is a continuation-in-part of U.S. patent application Ser. No. 18/212,621 filed Jun. 21, 2023, which is a divisional of U.S. patent application Ser. No. 17/720,143 filed Apr. 13, 2022 (now U.S. Pat. No. 11,771,304), which is a continuation-in-part of U.S. patent application Ser. No. 17/521,397 filed Nov. 8, 2021 (now U.S. Pat. No. 11,980,342), which claim priority to U.S. Provisional Appln. Ser. Nos. 63/176,307 filed Apr. 4, 2021; 63/112,739 filed Nov. 12, 2020; 63/113,960 filed Nov. 15, 2020; 63/118,617 filed Nov. 25, 2020; 63/128,105 filed Dec. 20, 2020; 63/138,528 filed Jan. 18, 2021; 63/295,913 filed Jan. 2, 2022; 63/299,829 filed Jan. 14, 2022; 63/299,960 filed Jan. 15, 2022; 63/302,563 filed Jan. 25, 2022; 63/303,690 filed Jan. 27, 2022; and 63/310,336 filed Feb. 15, 2022.

This application claims priority to and incorporates by reference U.S. Provisional Appln. Ser. No. 63/672,949 filed Jul. 18, 2024, U.S. Provisional Appln. 63/683, 198, filed Aug. 14, 2024, U.S. Provisional Appln. 63/685,682, filed Aug. 21, 2024, U.S. Provisional Appln. 63/693,589, filed Sep. 11, 2024, U.S. Provisional Appln. 63,713,926, filed Oct. 20, 2024, U.S. Provisional Appln. 63/718,614, filed Nov. 9, 2024, U.S. Provisional Appln. 63/734,073, filed Dec. 16, 2024, U.S. Provisional Appln. 63/743,649, filed Jan. 10, 2025, U.S. Provisional Appln. 63/756,997, filed Feb. 11, 2025, U.S. Provisional Appln. 63/766,315 filed Mar. 3, 2025.

This application incorporates by reference the entirety of the foregoing non-provisional and provisional patent applications and claims the benefit of the filing date of each as well as of the applications that they incorporated by reference, directly or indirectly, and the benefit of which they claim, including U.S. provisional applications, U.S. non-provisional applications, and international applications.

This patent application incorporates by reference each of the following U.S. patents and U.S. and international (PCT) patent applications:
Ser. No. 16/972,989 filed Dec. 7, 2020;
PCT/US21/50095 filed Sep. 13, 2021;
PCT/US2017/053171 filed Sep. 25, 2017
Ser. No. 17/835,624 filed Jun. 8, 2022;
Ser. No. 16/363,209, filed Sep. 25, 2017, now U.S. Pat. No. 11,832,797;
Ser. No. 17/843,217, filed Jun. 17, 2022
PCT/US16/18670 filed Feb. 19, 2016;
Ser. No. 14/913,867 filed Feb. 23, 2016, now U.S. Pat. No. 10,874,287;
PCT/US16/65396 filed Dec. 7, 2016;
Ser. No. 15/371,858 filed Feb. 20, 2018, now U.S. Pat. No. 9,895,048;
Ser. No. 15/462,331 filed Mar. 17, 2017, now U.S. Pat. No. 10,524,636;
Ser. No. 15/651,526 filed Jul. 17, 2017, now U.S. Pat. No. 10,278,563;
Ser. No. 15/855,532 filed Dec. 27, 2017, now U.S. Pat. No. 10,292,571;
PCT/US18/14880 filed Jan. 23, 2018;
Ser. No. 16/407,028 filed May 8, 2019, now U.S. Pat. No. 11,253,141;
Ser. No. 16/413,160 filed May 15, 2019, now U.S. Pat. No. 10,869,592;
Ser. No. 16/447,251 filed Jun. 20, 2019, now U.S. Pat. No. 11,013,141;
PCT/US20/38349 filed Jun. 18, 2020;
PCT/US20/46018 filed Aug. 12, 2020;
Ser. No. 17/122,282 filed Dec. 15, 2020;
Ser. No. 17/145,466 filed Jan. 11, 2021, now U.S. Pat. No. 11,395,579;
Ser. No. 17/370,575 filed Jul. 8, 2021;
Ser. No. 17/349,674 filed Jun. 16, 2021;
Ser. No. 17/573,095 filed Jan. 24, 2022;
PCT/US2017/053171 filed Sep. 25, 2017
Ser. No. 17/835,624 filed Jun. 8, 2022;
Ser. No. 16/363,209 filed Sep. 25, 2017, now U.S. Pat. No. 11,832,797;
Ser. No. 17/843,217 filed Jun. 17, 2022;
Ser. No. 17/362,043 filed Jun. 29, 2021, now U.S. Pat. No. 11,350,816;
Ser. No. 17/473,587 filed Sep. 13, 2021, now U.S. Pat. No. 11,330,973;
Ser. No. 17/745,526 filed May 16, 2022;
Ser. No. 17/521,397 filed Nov. 8, 2021;
Ser. No. 17/720,143 filed Apr. 13, 2022;
Ser. No. 18/212,621 filed Jun. 21, 2023;
Ser. No. 17/941,884 filed Sep. 9, 2022;
Ser. No. 17/835,624 filed Jun. 8, 2022, now U.S. Pat. No. 11,684,248
Ser. No. 18/083,209 filed Dec. 16, 2022;
Ser. No. 18/113,395 filed Feb. 23, 2023;
Ser. No. 18/211,486 filed Jun. 19, 2023;
Ser. No. 18/233,282 filed Aug. 11, 2023;
Ser. No. 18/374,740 filed Sep. 29, 2023;
Ser. No. 14/913,867 filed Feb. 23, 2016, now U.S. Pat. No. 10,874,287;
Ser. No. 16/407,028 filed May 18, 2019, now U.S. Pat. No. 11,253,141;
Ser. No. 16/413,160 filed May 15, 2019, now U.S. Pat. No. 10,869,592;
Ser. No. 17/122,282 filed Dec. 15, 2020, now U.S. Pat. No. 11,844,498;
Ser. No. 15/371,858 filed Dec. 7, 2016, now U.S. Pat. No. 9,895,048;
Ser. No. 13/094,415 filed Apr. 26, 2011, now U.S. Pat. No. 9,649,014;
Ser. No. 15/462,331 filed Mar. 17, 2017, now U.S. Pat. No. 10,524,636;
Ser. No. 15/651,526 filed Jul. 17, 2017, now U.S. Pat. No. 10,278,563;
Ser. No. 15/855,532 filed Dec. 27, 2017, now U.S. Pat. No. 10,292,571;
Ser. No. 15/484,953 filed Apr. 11, 2017, now U.S. Pat. No. 10,426,320;
PCT/US2020/038349 filed Jun. 18, 2020, now U.S. Pat. No. 11,013,396;
Ser. No. 17/370,575 filed Jul. 8, 2021;
Ser. No. 18/209,947 filed Jun. 14, 2023;
Ser. No. 17/145,466 filed Jan. 11, 2021, now U.S. Pat. No. 11,395,579;
Ser. No. 16/664,082 filed Oct. 25, 2019, now U.S. Pat. No. 11,071,442;
Ser. No. 17/349,674 filed Jun. 16, 2021
PCT/US2020/046018 filed Aug. 12, 2020;
Ser. No. 17/583,095 filed Jan. 24, 2022;
Ser. No. 13/276,839 filed Oct. 19, 2011, now U.S. Pat. No. 8,702,594; and
Ser. No. 13/094,415 filed Apr. 26, 2011, now U.S. Pat. No. 9,649,014;
Ser. No. 18/982,803 filed Dec. 16, 2024;
Ser. No. 18/628,133 filed Apr. 5, 2024;
Ser. No. 18/622,471, filed Mar. 29, 2024;
Ser. No. 19/089,980 filed Mar. 25, 2025.

### FIELD

This patent specification relates to endoscopes and more specially to a new approach to enhancing benefits of portable, hand-held endoscopes.

### BACKGROUND

Clinicians use endoscopes to examine internal organs such as the bladder and the upper urinary tract, the uterus, and other internal organs. This can involve passing a cannula through the urethra into the bladder and then from the bladder into the ureter and possibly into a kidney, or passing the cannula to the uterus, or passing the cannula to another internal organ through a suitable passageway.

Disposable or partly disposable (single-use) endoscopes are gaining market share over traditional, multiple-use endoscopes by lessening the risk of cross-contamination and hospital acquired diseases, making it practical to perform procedures in a doctor's office in addition to clinics and hospitals, and reducing the overall cost of medical procedures by avoiding expenses associated the higher capital cost of traditional endoscopes and with sterilizing and maintaining traditional endoscopes that requires special equipment and personnel for such maintenance.

Known telesurgery, also known as remote surgery, relies on sophisticated robotic systems that surgeons control remotely. Reliable high-speed communication networks are crucial for transmitting real-time data and ensuring seamless control of robotic instruments. Maintaining a low latency (delay) in the communication link is essential for successful telesurgery as delays can affect the surgeon's ability to coordinate movements with the robot. The initial cost of robotic systems and high-speed telecommunication infrastructure can be a barrier to entry, especially for resource-limited settings, and makes it impractical for individual clinician offices to make the investment. For example, a telesurgery system “Da Vinci” is offered by Intuitive Surgical, Inc.—see https://www.intuitive.com/en-us/about-us/company.

Disposable or partly disposable hand-held endoscopes are much less expensive and much simpler to use, and have many other advantages over both traditional endoscopes and known telesurgery systems. Typically, a clinician such as a urologist or a gynecologist carries out a medical procedure by manually controlling the endoscope. The patents and applications incorporated by reference describe examples of such endoscopes.

The subject matter described or claimed in this patent specification is not limited to embodiments that solve any specific disadvantages or that operate only in environments such as those described above. Rather, the above background is only provided to illustrate one exemplary technological area where some embodiments described herein may be practiced.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims, and that components of one or more of the embodiments may be used in any other embodiments as needed or suitable. The term endoscope as used in this patent specification is intended to encompass a ureteroscope. The subject matter hereof will be described and explained with additional specificity and detail through the accompanying drawings in which:

FIG. 1 is a perspective view of a tele-endoscopy system, according to some embodiments.

FIG. 2 is a perspective view of a remote station of a tele-endoscopy system, according to some embodiments.

FIG. 3 is a perspective view of a tele-endoscopy system, according to some embodiments.

FIG. 4 is a perspective view of an endoscope useful in a tele-endoscopy system, according to some embodiments.

FIG. 5 is a perspective view from a different viewpoint of an endoscope useful in a tele-endoscopy system, according to some embodiments.

FIG. 6 is a perspective view of a reusable portion and a single-use portion of an endoscope useful in a tele-endoscopy system, according to some embodiments.

FIG. 7 is a perspective view of an endoscope useful in a tele-endoscopy system, according to some embodiments,

FIG. 8 is a perspective view of the endoscope of FIG. 7 taken from another viewpoint.

FIG. 9 illustrates an endoscope with motorized drives in a reusable portion to deflect a distal end of a camera and in a single-use portion to rotate and/or axially translate the cannula, according to some embodiments.

FIG. 10 illustrates a distal end of a cannula provides with temperature and pressure sensors and with a position indicating member.

### SUMMARY OF THE DISCLOSURE

A hand-held, low-cost endoscope is incorporated in a tele-endoscopy system such that a clinician at a local station controls the operation of a remote endoscope to image or treat internal organs such as the bladder, the uterus, or the upper urinary tract while viewing real-time patient images that the remote endoscope takes with a cannula tip camera and with a handle camera. The remote endoscope can still be operated individually to carry out or complete the procedure, for example if the link to the local station degrades or if there is no video link.

The tele-endoscopy described in this patent specification fills a gap between known telesurgery with expensive equipment and critical dependence on a high-quality communication network and individually used hand-held endoscopes that have a much lower cost and other advantages. The system is particularly user-friendly and dependable as it uses simpler components that are less likely to develop issues. The local station interfaces with a clinician are intuitive, closely mirroring the endoscope motions at the remote station. The endoscopes in the system can be cystoscopes, hysteroscopes, ureteroscopes, or other scopes.

The new system has a robotic mode in which a remote endoscope is mounted to a robotic table. Motorized drives in the remote endoscope rotate and deflect a camera-tipped cannula as commanded with a manually operated cannula control by a clinician at a local station that has no direct view of the remote endoscope. In addition, the clinician at the local station commands motion of a robotic table to which the remote endoscope is mounted for motion along and about a vertical axis and for tilting about a horizontal axis. Typically, a health professional such as a physician assistant (PA) or a nurse at the remote station hand-manipulates the remote endoscope's cannula to introduce it into a passageway leading to the internal cavity of interest and a clinician at the local station then takes control of the remote endoscope. The robotic table motion is primarily to position the remote endoscope in space while finer cannula motion is controlled with cannula control at the local station and, if needed, at the remote station. The remote endoscope can be unmounted from the robotic table so the PA can hold it to introduce the cannula in the passageway or carry out some or all of the imaging/treatment procedure using the remote endoscope. In special cases, the entire procedure could be carried out by the clinician controlling the robotic table and the remote endoscope from the local station. The remote endoscope remains fully capable of performing the medical or other procedure with the remote endoscope, including rotating and deflecting the cannula, for example if a link to the local station degrades.

The new system has an alternative mode—a hybrid mode in which a health professional at the remote station inserts the cannula in the patient and continues to hold the remote endoscope while a clinician at the local station controls the remote endoscope's cannula motions relative to the handle while viewing images from the handle camera and the cannula tip camera of the remote endoscope. This can eliminate the need for a robotic table and can work well even when the communication link quality is not high as latency is not as critical as with known telesurgery. Both the local and the remote station still display the same images taken with the remote endoscope, but departures from real time image and command transmission can be less critical.

The new system has yet another alternative mode—a manual mode in which a health professional at the remote station uses the remote endoscope to carry out a medical procedure with only voice contact with a clinician. If the video link fails or no video is available so the clinician cannot see images from the remote endoscope, but voice contact is available, the health professional and the remote station can use the remote endoscope with voice guidance from the local station. If not even voice contact is available, the health professional can still use the remote endoscope by manually operating its control over cannula rotation and deflection and other endoscope functions.

The new approach enables a change from one of the modes to another during a medical procedure. For example, if the communication link is unable to transmit real time images and commands and or only allows transmission at high latency, the new system can change from robotic to hybrid mode. If the link does not allow any transmission of images or fails to such a state, the new tele-endoscopy system can change to manual mode. Conversely, changes in the other direction can be available.

The local and remote stations can be very far from each other, even in different continents, but also can be very close to each other such as in the same hospital or clinic and even in the same room (for example for training of health professionals).

The endoscope at the remote station can be motorized such that inputs from a manually operated interface at either the local station or the remote station can both (i) rotate a cannula of a single-use portion relative to a reusable portion to which the single-use portion is detachably mounted and (ii) deflect a distal end of the cannula relative to a reusable portion. In the manual mode, the health professional at the remote station can perform the medical procedure independently, by moving the endoscope relative to the patient and controlling motorized cannula rotation and deflection with an interface such as a joystick on the remote endoscope. In robotic mode, the robotic table positions the remote endoscope in space relative to the patient and the remote endoscope's motorized drives rotate and deflect the cannula without depending on the robotic table. In case the remote endoscope further has a motorized drive to translate the cannula relative to the reusable portion along the cannula axis, the robotic table can be used for coarse motion while the motorized drive in the endoscope can be used for fine translation along the cannula axis. As noted, initial insertion of the remote endoscope's cannula into a passageway leading to the internal cavity or organ of interest can be done manually at the remote station or with the robotic table.

The local and remote stations can use duplicate endoscopes, differentiating the new tele-endoscopy approach from known telesurgery where the equipment at two stations differs significantly. In the new approach, the equipment at the local station can be simplified by equipping the local station with only the cannula control of an endoscope to control the motorized drives of the remote endoscope.

According to some embodiments, a tele-endoscopy system in which a local station with a local endoscope is linked with a remote station with a remote portable endoscope that is configured to be hand-held for an endoscopic procedure, wherein: at least the remote endoscope has (i) internal motorized drives (ii) a reusable portion with a handle and a distal-facing handle camera and (iii) a single-use portion with a cannula extending distally from the handle along a cannula axis, tipped with a cannula camera, and mounted to rotate relative to the reusable portion about the cannula axis and to deflect a distal end from the cannula axis; each of the local and the remote endoscope has a manually operated cannula control configured to generate cannula control signals commanding the motorized drives to rotate and deflect the remote endoscope's cannula; each of the local and remote endoscopes has a display configured to show images taken with the remote endoscope's handle camera and cannula tip cameras; a robotic table at the remote station and a manually operated table control at the local station configured to generate table control signals commanding the robotic table to translate, rotate, and tilt the remote endoscope; and the remote endoscope is configured to independently carry out the endoscopic procedure using the remote endoscope's cannula control to rotate and deflect the remote endoscope's cannula if a link with the local station degrades below a threshold.

According to some embodiments, the tele-endoscopy system can further include one or more of: (a) the local endoscope is a duplicate of the remote endoscope; (b) the remote endoscope's motorized drives include an electric motor in the handle to deflect the cannula's distal end and an electric motor in the single-use portion to rotate the cannula; (c) the single-use portion further has an electric motor configured to axially translate the cannula along the cannula axis relative to the handle as commended by the manually operated cannula control; (d) the remote endoscope's handle camera has a field of view that includes the remote endoscope's cannula and external anatomy of a patient; (e) the handle camera is configured to provide a stereo view; (f) the tip camera comprises two image sensors, each configured to image light in a respective wavelength range; (g) at least the remote endoscope's cannula has sensors providing the local station with temperature and pressure data at the remote endoscope's cannula; (h) at least the remote is configured with a 3D positioning sensor to provide the local station with data indicating the position and orientation of the distal end of the remote endoscope's cannula; (i) the local station has a stand mounting the manually operated robotic table control and the local endoscope's manually operated cannula control; (j) the robotic table further has an stand configured to support medication; (k) the display at each of the local and the remote stations is mounted to the respective endoscope; (l) the local station has a display that is in addition to and is larger than the endoscope-mounted display and is configured to show images taken with the remote endoscope's handle and tip cameras; and (m) each of the local and the remote station has a wireless and/or a wired connection with a network backbone for communicating with each other.

According to some embodiments a tele-endoscopy system with a local endoscope operated by a clinician who has no direct view of a patient and a remote hand-held endoscope that provides patient images to the local endoscope and is controlled by the local endoscope, wherein: at least the remote endoscope has (i) internal motorized drives (ii) a reusable portion with a distal-facing handle camera and (iii) a single-use portion with a cannula extending distally from the handle along a cannula axis, tipped with a cannula camera, and mounted to rotate relative to the reusable portion about the cannula axis and to deflect a distal end from the cannula axis; each of the local and the remote endoscope has a manually operated cannula control configured to generate cannula control signals commanding the motorized drives to rotate and deflect the remote endoscope's cannula; each of the local and remote endoscopes has a display configured to show images taken with the remote endoscope's handle and cannula tip cameras; and the remote endoscope is configured to independently carry out the endoscopic procedure using the remote endoscope's cannula control to rotate and deflect the remote endoscope's cannula if a link with the local endoscope degrades below a threshold.

The tele-endoscopy system described in the immediately preceding paragraph can further include one or more of: (a) the motorized drives include an electric motor in the cannula configured to rotate the cannula and an electric motor in the reusable portion configured to deflect the cannula's distal end; (b) the single-use portion further includes an electric motor configured to axially translate the cannula along the cannula axis relative to the reusable portion; and (c) the motorized drives are further configured to cause axial translation of the cannula along the cannula axis relative to the handle and the manually operated interface is further configured to command the motorized drives to cause the axial translation.

According to some embodiments, a tele-endoscopy method of imaging or treating an internal cavity reachable through a passage, comprises: at a remote station, inserting a cannula of a remote endoscope through the passage such that the cannula enters the cavity and a handle from which the cannula extend along a cannula axis is outside the passage; taking stereo images of a distal field of view of an area that includes the passage and the cannula with a stereo camera mounted to the handle and images of the cavity with a cannula tip camera; displaying the images at a local station that has no direct view of the remote endoscope or the passage; and configuring motorized drives in the remote endoscope to rotate the cannula relative to the handle about the cannula axis and to deflect a distal end of the cannula relative to the cannula axis endoscope's cannula in response to both a manually operated cannula control at the local station and a manually operated cannula control at the remote endoscope.

The tele-endoscopy method can further include one or more of: (a) mounting the remote endoscope to a robotic table for translation along and rotation about a vertical axis and tilting about a horizontal axis relative to a frame of reference in response to a table control at the local station; and (b) making the local and remote endoscopes the same.

### DETAILED DESCRIPTION OF EXAMPLES

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims, and that components of one or more of the embodiments may be used in any other embodiments as needed or suitable. The term endoscope as used in this patent specification is intended to encompass a ureteroscope. The subject matter hereof will be described and explained with additional specificity and detail through the accompanying drawings in which:

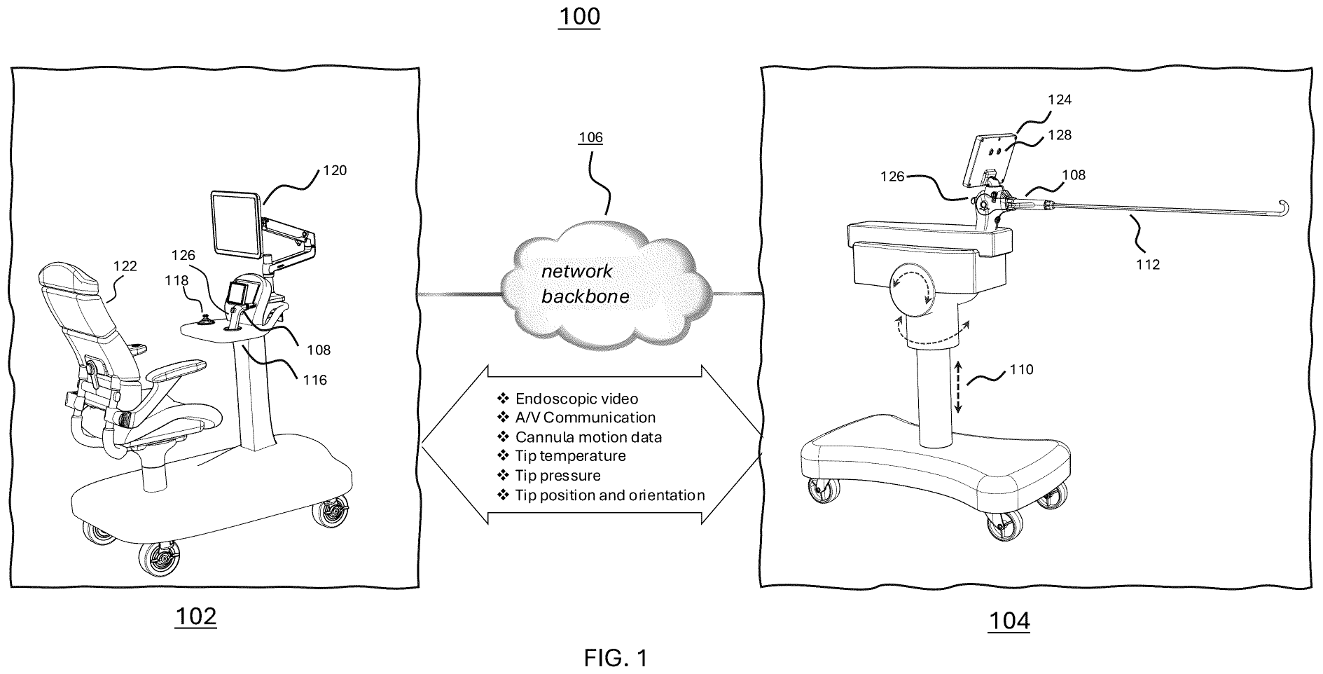

FIG. 1 illustrates a tele-endoscopy system 100 configured for operation in a robotic mode according to some embodiments. The system has a local station 102 communicating with a remote station 104 over a communication link or network backbone 106. Each of the stations has an endoscope 108. The two endoscopes can be identical although in some embodiments the endoscope at local station 102 can omit some of the components of the endoscope at remote station 104. The local station sometimes is called the clinician or the MD station, and the remote station sometimes is called the patient unit (PU). Typically, a clinician such as a urologist is at the local station and a health professional such as a nurse or a PA is at the local station although a clinician can be at each of the stations.

At remote station 104, endoscope 108 is mounted to a robotic table 110 that is configured to move the endoscope by motorized drives in four motions as indicated by arrows: (i) up and down along a vertical axis, (ii) in rotation about the vertical axis, (iii) in tilting about a horizontal axis, and (iv) in translation along the long axis of cannula 112. Robotic table 110 preferably has locking wheels 114 so it can be rolled to a desired position relative to a frame of reference such as a room and a patient for a medical procedure such as to examine or treat the bladder, upper urinary tract, uterus, or another organ. Local station 102 has a duplicate endoscope 108 that can be mounted on a stand 116 that additionally mounts a table control 118 such as a joystick and a larger display 120. Local station 102 can additionally include chair 122 for a clinician who operates table control 118 and endoscope 108. As described further below, each of endoscopes 108 has a reusable portion to which a single-use portion mounts detachably, a display 124, and a manually operated endoscope control 126 such as a joystick. The single-use portion of each endoscope 108 has a cannula 112 with a tip camera at its distal end, and each reusable portion has a handle camera 128 that can be at the back of display 124 and has a field of view that includes the cannula and the patient.

Communication link 106 transmits to local station 102 images taken with endoscope 108 at remote station 104 and transmits commands from local station 102 to remote station 104 from control 126 at local station 102. Bidirectional link 106 can further transmits other information such as commands for motions of table 110 entered by operating table control 118 at local station 102, feedback from table 110 about motions or positions of the table, audiovisual (A/V) data between people and equipment at the two stations, information from sensors (if any) at remote station 104 such as sensors for pressure and temperature in cannula 112 of endoscope 108 at the remote station, patient information, position information about the cannula at the remote station, etc.

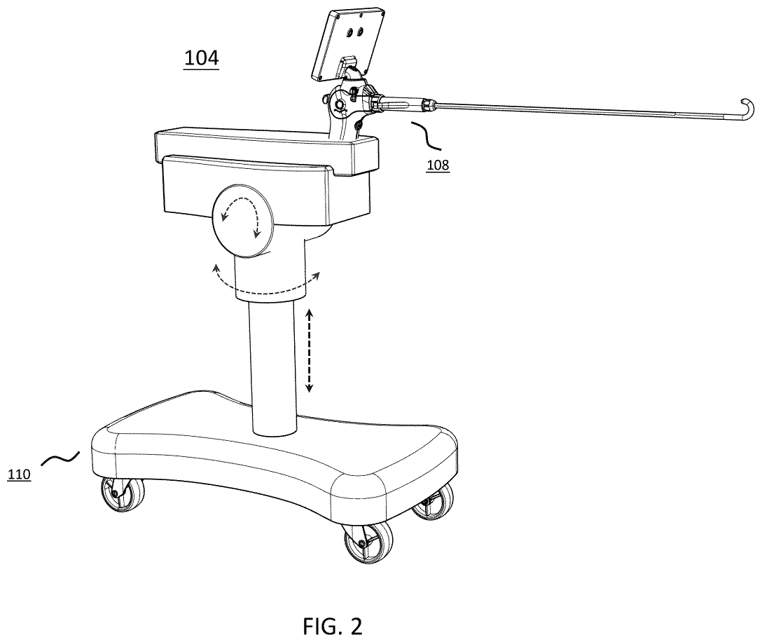

FIG. 2 is a perspective view of robotic table 110 with endoscope 108 mounted to it and with arrows indicating motions of the table and the remote endoscope relative to a frame of reference such as a room.

In robotic mode of the new tele-endoscopy system, a clinician such as a urologist or a gynecologist is at local station 102. A like clinician, or a health professional such a nurse, or even a person with less training in special cases, is at remote station 104, with a patient. The person at the remote station assembles endoscope 108 by opening a sterile package containing a new single-use portion and attaches it to a reusable portion to assemble endoscope 108, mounts endoscope 108 to robotic table 110 and positions the robotic table relative to a patient such that the distal tip of cannula 112 is close to and points to a passageway such as the patient's urethra. Endoscope 108 at remote station 104 transmits over link 106 images both from a tip camera at the distal end of cannula 112 and from a handle camera with a field of view that includes the distal tip of cannula 112 and the patient's pertinent external anatomy to local station 102 for display at local station 102 on display 124 of endoscope 108 at local station 102 and at larger display 120. A display 124 at remote station 104 shows the same images. The health professional at remote station 102 can manually insert the tip of cannula 112 into the passageway and can continue insertion until the cannula tip has reached a desired location in an internal cavity such as the bladder, while the clinician at local station 102 is observing images from the tip and handle cameras at the remote station. Alternatively, some or all of the canula insertion can be commanded by the clinician at local station 102 by operating table control 118 such as a joystick at local station 102. During the insertion and after the cannula tip has reached a desired organ such as the bladder, the clinician at local station 102 operates interface 126 such as joystick of endoscope 108 at local station 102 to command rotation of cannula 112 at remote station 104 about axis A relative to the reusable part of endoscope 108 and relative to robotic table 110 and deflection of a distal end of cannula 112 at remote station 104 from the cannula's axis, while continuing to observe at local station 102 images taken with endoscope 108 at remote station 104. If endoscope 108 has additional tools such as a grasper, an injection needle, or a stent insertion and removal, and irrigation or suction, the clinician at local station 102 can command endoscope 108 at remote station 104 to carry out appropriate steps using such functionalities with a manually operating interface at local station 102. The clinician at local station 102 can command withdrawal of endoscope 108 from the patient by commanding robotic table 110 with commands from table control 118, or the health professional at the remote station can manually withdraw endoscope cannula 112 from the patient.

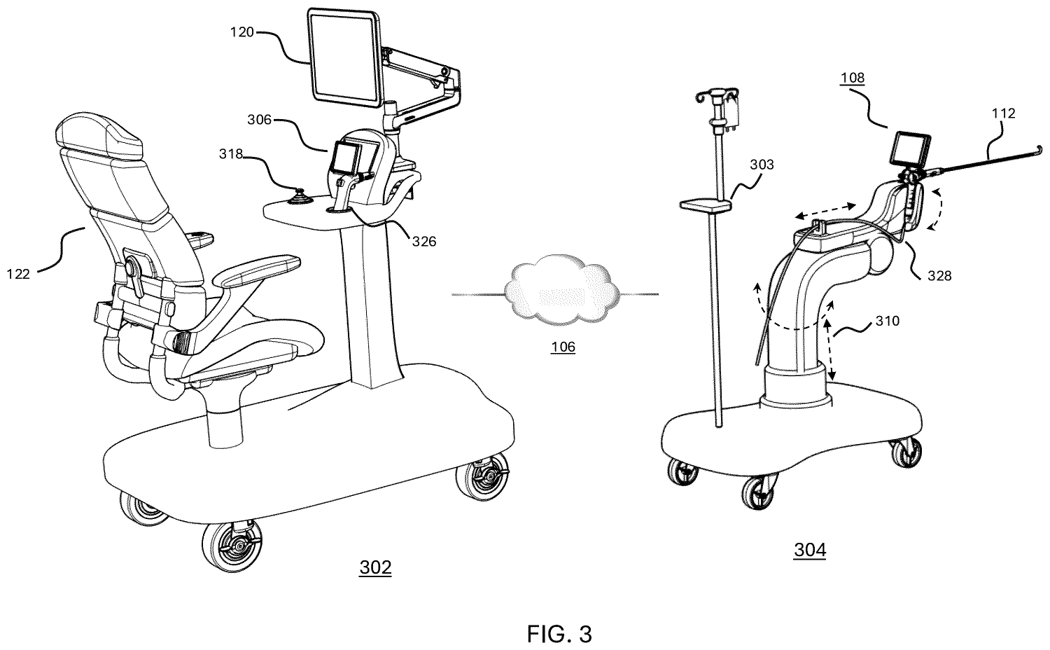

FIG. 3 is a perspective view of a tele-endoscopy system that has a local station 302 and a remote station 304 communicating through link 106 such as the Internet, according to some embodiments. Local station 302 is otherwise like local station 102 but has a manually operated cannula control 326 that duplicates the action and function of cannula control 126 but is not attached to an endoscope. Cannula control 326 is configured to generate the same electrical output data as interface 126 generates to control the motorized drives of remote endoscope 108. In addition, local station 102 has a touch panel 306 configured to display images such as from handle cameras 128 of endoscope 108 at remote station 304. Larger display 120 can display images from the tip camera of endoscope 108 at remote station 104 and/or additional images such as from handle camera 128 at remote station 104 and patient data. Remote station 304 is otherwise like remote station 104 but, as indicated by arrows in FIG. 3, robotic table 310 is configured to mount endoscope 108 and to move the support for the endoscope (i) in rotation about a vertical axis, (ii) in translation along the vertical axis, (ii) in sliding endoscope 108 in translation along its cannula's long axis, and (iv) in pivoting endoscope 108 about a horizontal axis transverse to the cannula's long axis. Remote station 304 includes a cable 328 connecting endoscope 108 to link 106 for a wired transmission of data between stations 102 and 104 instead of or in addition to wireless transmission. Each of local station 302 and remote station 104 includes a wired or wireless connection to a link such as the Internet, for example through a router at each station. In addition, remote station 304 has stand 303 for supporting a medication source such as an IV solution bag.

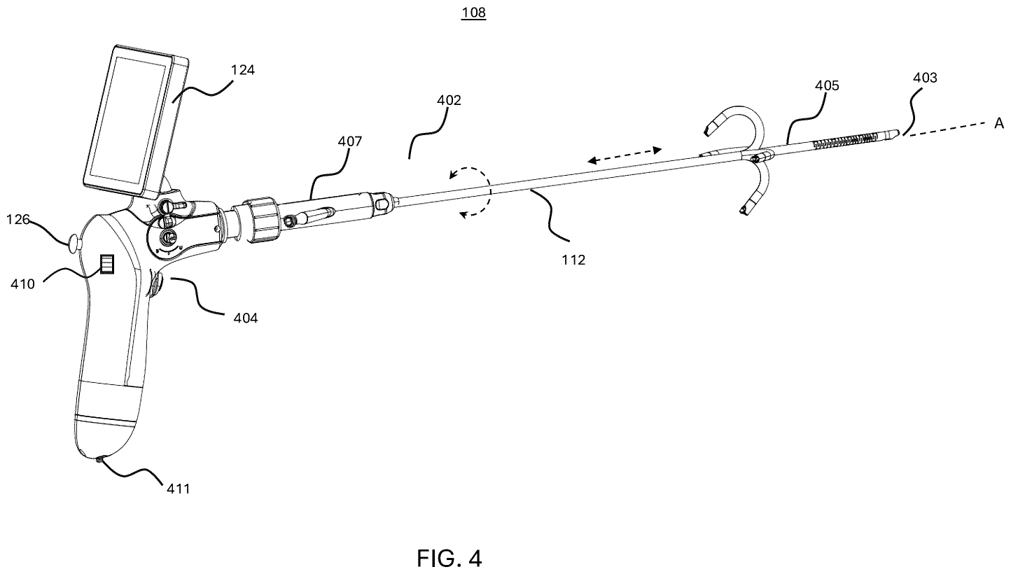

FIG. 4 illustrates a compact, hand-held, robotic assisted endoscope 108 that can be used in the tele-endoscopy systems of FIGS. 1 and 3 according to some embodiments. Endoscope 108 comprises a single-use portion 402 that includes a cannula 112 with a camera and light source module 403 at its distal end and a reusable portion 404 that includes a handle 406 and a display 124 that typically displays images acquired with a tip camera in module 403 and/or other information such as patient and procedure identification and other images. Module 103 can comprise a single image sensor or two or more image sensors that can serve as independent cameras to provide stereo or 3D views or each can image a different range of wavelengths. As indicated by arrows, cannula 112 is configured to rotate about the cannula's long axis relative to reusable portion 404 and to translate along the long axis relative to reusable portion 404. Distal end 405 of cannula 112 is configured to deflect (angulate or bend) relative to the long axis A of cannula 112. Handle 406 typically includes manually operated interface controls such as buttons, joystick and/or touch pad 126 serving as a cannula control through which a clinician can control deflection, rotation and/or translation of the distal and/or other functions of the single-use portion, for example with the thumb of the hand holding handle 406. Distal end 405 of single-use portion 402 can articulate (angulate or deflect) to assume positions such as illustrated, in addition to a position in which it is straight along the long axis of cannula 112. The illustrated robotic-assistance endoscope 108 augments human operator performance by combining human skill with the precision and artificial intelligence of robotic facilities, as described in more details in co-pending U.S. Patent Appln. Ser. No. 17/941,884, filed Sep. 9, 2023, and published as US 2023/0117151 A1, hereby incorporated by reference. Handle 406 can include a wireless transmitter/receiver 410 to receive images and other data from remote station 104 and transmit control signals from cannula control 126 of local station 102 to remote station 104.

The electrical outputs of cannula control 126 and table control 118 can be in digital form, in which case they can be transmitted to and used to control rotation, deflection, and axial translation directly in digital form to remote station 104. If the outputs are in analog form, the local and remote stations can include known converters between analog and digital formats for digital format transmission. The images that the remote station's cameras take typically are in digital format and can be transmitted directly in that format or converted to a specific desired format for transmission and display.

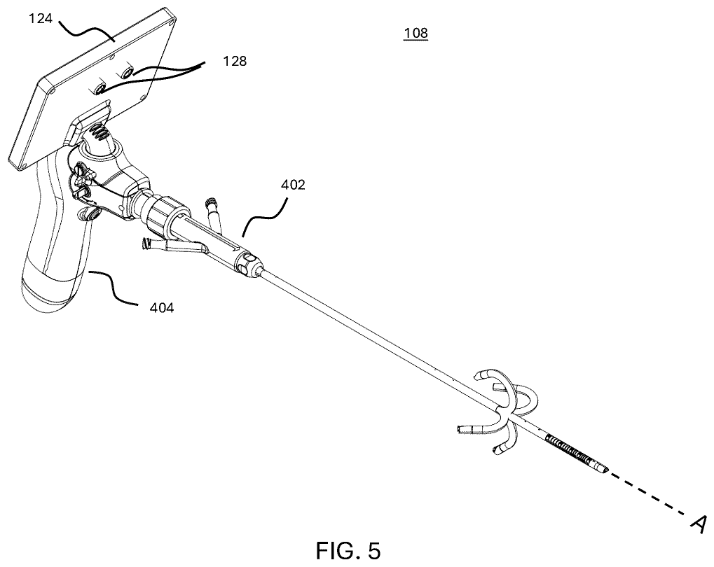

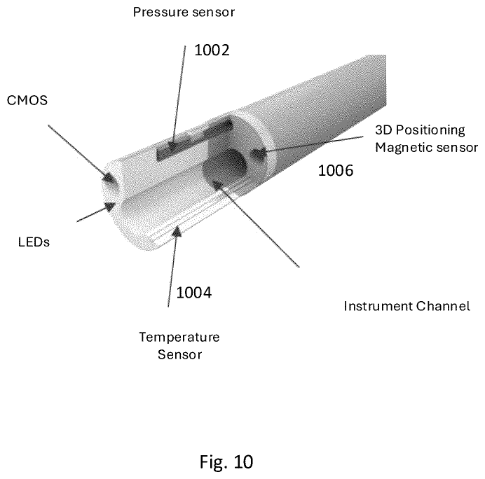

FIG. 5 is a perspective view of endoscope 108 shown in FIG. 4, taken from a different viewpoint to show forward-looking handle cameras 128 at the back of display 124. Endoscope 108 used in the tele-endoscopy systems of FIGS. 1 and 3 can be as illustrated in FIGS. 4 and 5 or can be another of the endoscopes shown and described in the patents and applications incorporated by reference herein or can comprise combinations of their features or can be a like variation thereof. In the example of FIG. 5e distally (forward-facing) cameras 128 have a field of view that includes distal end 405 of reusable portion 402, as also shown as items 1002 in FIG. 10 of application Ser. No. 17/941,884 (US 2023/0117151 A). Module 403 at the distal end of cannula 112 can comprise one or more cameras that selectively image different ranges of light wavelengths and the light source such as LEDs in module 403 can selectively emit light in desired different wavelength ranges. Endoscope 108 can include permanently mounted surgical devices such as a grasper, an injection needle, etc. and can include one or more working channels through which surgical devices can be inserted to reach an inner region of a patient (not shown) and can include fluid channels through which fluids can be introduced into or withdrawn from a patient, as described in the patents and applications incorporated by reference.

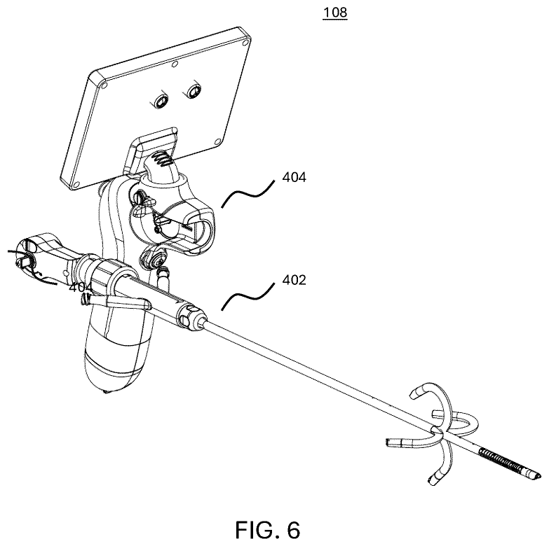

FIG. 6 are perspective view of single-use portion 402 and reusable portion 404 before they are assembled into endoscope 108.

Endoscope 108 includes motorized drives configured to cause (i) axial rotation of cannula 112 about axis A relative to reusable portion 404 and handle 406 and (ii) deflection of the cannula's distal portion 405 away from axis A in at least two angular directions. The motorized drives include an electric motor in reusable portion 404 operatively coupled with distal portion 405 via cables to deflect portion 405 away from axis A and an electric motor inside a hub 407 of single-use portion 402 operatively coupled to cannula 112 to rotate the cannula about axis A. Manually operated cannula control 126 operatively couples with the motorized drives to cause the axial rotation and the deflection of cannula 112. Such drives are described in more detail in U.S. patent application Ser. No. 18/083,209 filed Dec. 16, 2022 and published as US 2023/0128803 A, incorporated by reference herein. Handle 406 includes electronics coupled with (i) imaging module 403, which has a cannula tip camera, and (ii) handle cameras 128, to process images taken with the tip and handle cameras and display them on the screen of display 124. Endoscope 108 includes an internal wireless facility 209 such as a WiFi module and/or a cable connection to an interface to the Interned such as router.

Another example of an endoscope 108 that can be used in the tele-endoscopy system described above has self-contained motorized drives to carry out all three motion of the cannula relative to the handle-rotation, deflection, and translation. Using this endoscope can avoid a need for a robotic table in the first, robotic mode as the health practitioner at remote station 104 can hold endoscope 108 while the clinician at local station 102 commands cannula rotation, deflection, and translation at remote station 104 through link 106.

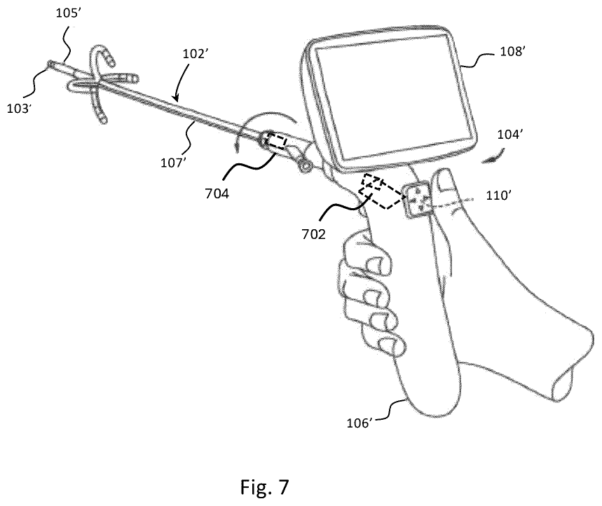

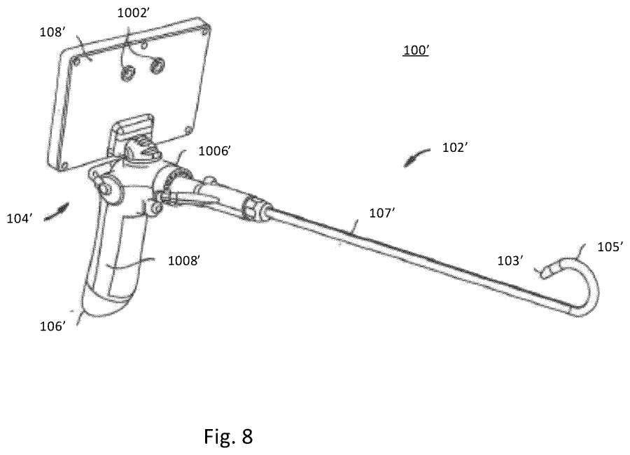

FIGS. 7 and 8 are perspective views of such an endoscope 108 taken from different viewpoints and are copies of FIGS. 1 and 10, respectively, of prior U.S. patent application Ser. No. 17/941,884 filed Sep. 9, 2022 and published as US 2023/0117151 A1, incorporated by reference herein. Text describing these FIGs. is copied below but should be understood in the context of the entire contents of the prior application. FIG. numbers 1 and 10 have been changed to FIG. numbers 7 and 8 in the copied text below, and primes have been added to each reference numeral in the copied text to differentiate from otherwise like reference numerals used in FIG. 1-6 of this patent specification. Numbers of other FIGs. referred in the copied text have an added prime to differentiate from FIG. numbers in this patent specification. A schematic illustration of internal motorized drives 702 and 704 is added.

FIG. 7 illustrates a compact, hand-held, robotic assisted endoscopic system according to some embodiments. Endoscope 100′ comprises a single-use portion 102′ that includes a cannula 107′ with a camera and light source module 103′ at its distal end thereof and a reusable portion 104′ that includes a handle 106′ and a display 108′ that typically displays images acquired with the camera and/or other information such as patient and procedure identification and other images. Module 103′ can comprise two or more image sensors that can serve as independent cameras to provide stereo or 3D views. As indicated by arrows, cannula 107′ is configured to rotate and translate relative to reusable portion 104′ and a distal part 105′ of cannula 107′ is configured to angulate relative to a long axis of the cannula 107′. Handle 106′ typically includes controls such buttons, joystick and/or touch pad 110′ through which the user can control angulation, rotation and/or translation of the distal and or other parts of the single-use portion, for example with the thumb of the hand holding handle 106′. Distal part 105′ of single-use portion 102′ can articulate to assume positions such as illustrated in addition to being straight along the long axis of cannula 107′. The illustrated robotic-assistance endoscope augments human operator performance by combining human skill with the precision and artificial intelligence of robotic facilities, as described in more details below.

Endoscope 100′ can be as illustrated in FIG. 1′ or can be any one of the endoscopes shown and described in said patents and applications incorporated by reference herein or can comprise combinations of their features, or can be the endoscope without a display shown in FIG. 2′, or a like variation thereof. Display 108′ can have one or more distally-or forward-facing cameras FCC whose field or view includes distal end 105′ of reusable portion 102′, as discussed in more detail further below. Module 103′ at the distal end of cannula 107′ can comprise one or more cameras that selectively image different ranges of light wavelengths and the light source such as LEDs in module 103′ can selectively emit light in desired different wavelength ranges. Endoscope 100′ can include permanently mounted surgical devices such as a grasper, and injection needle, etc. and, can include a working channel through which surgical devices can be inserted to reach object 301′, and can include fluid channels through which fluids can be introduced into or withdrawn from object 301, as described in said patents and applications incorporated by reference herein.

FIG. 8 illustrates using one or more step motors to derive CamPose relative to HandlePose. Endoscope 100′ in the example includes two spaced-apart, forward-facing cameras (FFC) 1002′ with respective light sources at the distally facing side of display 108′. Digital step motors 1006′ inside reusable portion 104′ drive rotation and translation of cannula 107′ relative to handle 106′ and deflection or angulation of the distal part 105′ of cannula 107′. The arrangement of FIG. 10′ can be used as endoscope 100′ in the system of FIG. 5, or as a stand-alone arrangement. FFC 1002′ view cannula 107′, including its distal part 105′. Step motors 1006′ supply motor step signals to a processor 1008′ in reusable portion 104′ that is configured to determine, from a count of steps of the respective motors, the position and/or orientation of cannula 107′, including its distal part 105′ and tip. FFC 1002′ generate real time images of cannula 107′ and its distal portion 105′ and tip that also are fed to processor 1008′, which is configured to correlate these images with step motor counts to determine CamPose relative to reusable portion 104.′ If HandlePose is desirable, it can be determined as discussed above for other examples, and from that CamPose can be determined relative to a selected frame of reference in addition to relative to handle 106′.

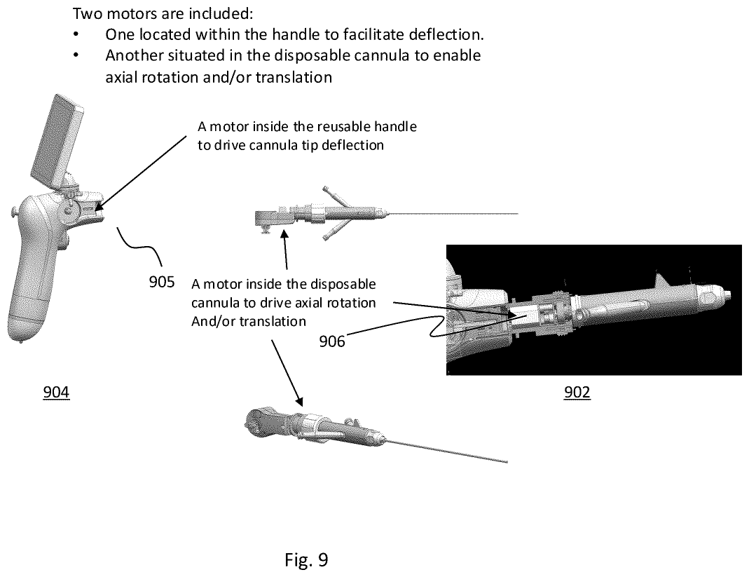

FIG. 9 illustrates a single-use portion 902 of an endoscope that has an internal electric motor 906 configured to rotate the cannula about axis A and/or translate the cannula along axis A relative to a reusable portion 904 that has an internal electric motor 905 configured to deflect the cannula's distal end. The cannula shown in FIG. 9 can be used as cannula 112 in the endoscopes described above.

FIG. 10 illustrates a portion of a cannula 112 that has a pressure sensor 1002, a temperature sensor 1004, and a magnetic sensor 1006, in addition to the indicated functional (working) channel and channels for CMOS camera and LED lighting. At least endoscope 108 at remote station 104 can include these sensors and electronics configured to transmit sensor readings to local station 104, and the local station can be configured to display these readings in essentially real time for the clinician at the local station to use in controlling the remote endoscope. Further, magnetic sensor 1006 can be used for 3-dimensional positioning of cannula 108 and its tip camera.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

What is claimed is:

1. A tele-endoscopy system in which a local station with a local endoscope is linked with a remote station with a remote portable endoscope that is configured to be hand-held for an endoscopic procedure, wherein:
at least the remote endoscope has (i) internal motorized drives (ii) a reusable portion with a handle and a distal-facing handle camera and (iii) a single-use portion with a cannula extending distally from the handle along a cannula axis, tipped with a cannula camera, and mounted to rotate relative to the reusable portion about the cannula axis and to deflect a distal end from the cannula axis;
each of the local and the remote endoscope has a manually operated cannula control configured to generate cannula control signals commanding the motorized drives to rotate and deflect the remote endoscope's cannula;
each of the local and remote endoscopes has a display configured to show images taken with the remote endoscope's handle camera and cannula tip cameras;
a robotic table at the remote station and a manually operated table control at the local station configured to generate table control signals commanding the robotic table to translate, rotate, and tilt the remote endoscope; and
the remote endoscope is configured to independently carry out the endoscopic procedure using the remote endoscope's cannula control to rotate and deflect the remote endoscope's cannula if a link with the local station degrades below a threshold.

2. The tele-endoscopy system of claim 1, in which the local endoscope is a duplicate of the remote endoscope.

3. The tele-endoscopy system of claim 1, in which the remote endoscope's motorized drives include an electric motor in the handle to deflect the cannula's distal end and an electric motor in the single-use portion to rotate the cannula.

4. The tele-endoscopy system of claim 1, in which the single-use portion further has an electric motor configured to axially translate the cannula along the cannula axis relative to the handle as commended by the manually operated cannula control.

5. The tele-endoscopy system of claim 1, in which the remote endoscope's handle camera has a field of view that includes the remote endoscope's cannula and external anatomy of a patient.

6. The tele-endoscopy system of claim 1, in which the handle camera is configured to provide a stereo view.

7. The tele-endoscopy system of claim 1, in which the tip camera comprises two image sensors, each configured to image light in a respective wavelength range.

8. The tele-endoscopy system of claim 1, in which at least the remote endoscope's cannula has sensors providing the local station with temperature and pressure data at the remote endoscope's cannula.

9. The tele-endoscopy system of claim 1, in which at least the remote is configured with a 3D positioning sensor to provide the local station with data indicating the position and orientation of the distal end of the remote endoscope's cannula.

10. The tele-endoscopy system of claim 1, in which the local station has a stand mounting the manually operated robotic table control and the local endoscope's manually operated cannula control.

11. The tele-endoscopy system of claim 1, in which the robotic table further has an stand configured to support medication.

12. The tele-endoscopy system of claim 1, in which the display at each of the local and the remote stations is mounted to the respective endoscope.

13. The tele-endoscopy system of claim 1, in which the local station has a display that is in addition to and is larger than the endoscope-mounted display and is configured to show images taken with the remote endoscope's handle and tip cameras.

14. The tele-endoscopy system of claim 1, in which each of the local and the remote station has a wireless and/or a wired connection with a network backbone for communicating with each other.

15. A tele-endoscopy system with a local endoscope operated by a clinician who has no direct view of a patient and a remote hand-held endoscope that provides patient images to the local endoscope and is controlled by the local endoscope, wherein:
at least the remote endoscope has (i) internal motorized drives (ii) a reusable portion with a distal-facing handle camera and (iii) a single-use portion with a cannula extending distally from the handle along a cannula axis, tipped with a cannula camera, and mounted to rotate relative to the reusable portion about the cannula axis and to deflect a distal end from the cannula axis;
each of the local and the remote endoscope has a manually operated cannula control configured to generate cannula control signals commanding the motorized drives to rotate and deflect the remote endoscope's cannula;
each of the local and remote endoscopes has a display configured to show images taken with the remote endoscope's handle and cannula tip cameras; and
the remote endoscope is configured to independently carry out the endoscopic procedure using the remote endoscope's cannula control to rotate and deflect the remote endoscope's cannula if a link with the local endoscope degrades below a threshold.

16. The tele-endoscopy system of claim 15, in which the motorized drives include an electric motor in the cannula configured to rotate the cannula and an electric motor in the reusable portion configured to deflect the cannula's distal end.

17. The tele-endoscopy system of claim 15, in which the single-use portion further includes an electric motor configured to axially translate the cannula along the cannula axis relative to the reusable portion.

18. The tele-endoscopy system of claim 15, in which the motorized drives are further configured to cause axial translation of the cannula along the cannula axis relative to the handle and the manually operated interface is further configured to command the motorized drives to cause the axial translation.

19. A tele-endoscopy method of imaging or treating an internal cavity reachable through a passage, comprising:
at a remote station, inserting a cannula of a remote endoscope through the passage such that the cannula enters the cavity and a handle from which the cannula extend along a cannula axis is outside the passage;
taking stereo images of a distal field of view of an area that includes the passage and the cannula with a stereo camera mounted to the handle and images of the cavity with a cannula tip camera;
displaying the images at a local station that has no direct view of the remote endoscope or the passage;
configuring motorized drives in the remote endoscope to rotate the cannula relative to the handle about the cannula axis and to deflect a distal end of the cannula relative to the cannula axis endoscope's cannula in response to both a manually operated cannula control at the local station and a manually operated cannula control at the remote endoscope.

20. The tele-endoscopy method of claim 19, further including mounting the remote endoscope to a robotic table for translation along and rotation about a vertical axis and tilting about a horizontal axis relative to a frame of reference in response to a table control at the local station.

21. The tele-endoscopy method of claim 20, including making the local and remote endoscopes the same.
